(19) United States
(12) Patent Application Publication
(10) Pub. No.: US 2009/0198454 A1
(43) Pub. Date: Aug. 6, 2009

(54) STANDARD SAMPLE FOR TEST AND /OR CALIBRATION OF CIRCULAR DICHROISM DISPERSION METER AND UV-VISIBLE SPECTROPHOTOMETER

(75) Inventors: Yoshihisa Inoue, Osaka (JP); Victor Borovkov, Osaka (JP); Akio Wada, Tokyo (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: JAPAN SCIENCE AND TECHNOLOGY AGENCY, Kawaguchi-shi, Saitama (JP)

(21) Appl. No.: 12/064,786

(22) PCT Filed: Feb. 22, 2006
(86) PCT No.: PCT/JP2006/303131

§ 371 Date: Feb. 25, 2008

(30) Foreign Application Priority Data
Aug. 23, 2005 (JP) 2005-241784

(51) Int. Cl. G01J 1/10; G06F 19/00; C07D 487/22

(52) U.S. Cl. 702/30; 356/243.1; 540/145

(57) ABSTRACT

A principal object of the present invention is to provide a standard sample that allows accurate testing and calibration of a circular dichroism spectrometer, and which can also be applied to testing and calibration of a UV-visible spectrophotometer. The present invention relates to a standard sample for use in testing and/or calibrating a circular dichroism spectrometer and a UV-visible spectrophotometer, which standard sample comprising:
a chlorin dimer represented by Chemical Formula (I):

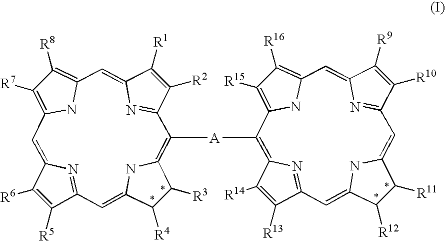

wherein A is CₙH₂ₙ (n is an integer of 0 or more); and R¹, R², R³R⁴, R⁵, R⁶, R⁷, R⁸, R⁹, R¹⁰, R¹¹, R¹², R¹³, R¹⁴, R¹⁵, and R¹⁶ are the same or different, and are each a hydrogen atom, a saturated hydrocarbon group, an aryl group that may be substituted, a heteroaryl group that may be substituted, or an aralkyl group that may be substituted; or
a metal chlorin dimer represented by Chemical Formula (II):

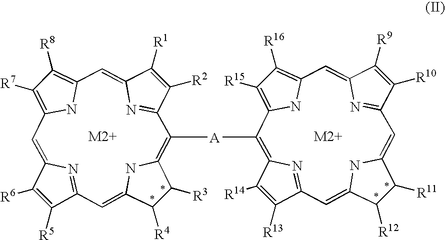

wherein M²⁺ is a divalent metal ion; and A, R¹, R², R³, R⁴, R⁵, R⁶, R⁷, R⁸, R⁹, R¹⁰, R¹¹, R¹², R¹³, R¹⁴, R¹⁵, and R¹⁶ are the same as defined above. Each of the chlorin dimer and the metal chlorin dimer exhibits at least two peaks in an ultraviolet to visible region of a circular dichroism spectrum and a UV-visible absorption spectrum thereof.

### TECHNICAL FIELD

The present invention relates to a novel standard sample for testing and/or calibrating a circular dichroism spectrometer and a UV-visible spectrophotometer.

### BACKGROUND ART

Optically active compounds attract attention as being very useful in the fields of pharmaceuticals, agriculture, foods, and the like. In synthesizing and identifying such an optically active compound, it is necessary to determine its absolute configuration. Circular dichroism spectrometers have previously been used to determine absolute configuration (Non-Patent Document 1).

In measuring an optically active compound using a circular dichroism spectrometer, it is necessary to test whether the measurement results provided by the circular dichroism spectrometer are correct or not, and, when necessary, to calibrate wavelengths and intensities. As a standard sample for such testing and calibration, an aqueous solution of ammonium d-10-camphorsulfonate has been used.

With this solution, however, the circular dichroism intensity at only one wavelength in the ultraviolet region can be observed. Therefore, testing and calibration cannot be performed with high accuracy simultaneously in a wide range of wavelengths between the ultraviolet and visible regions using ammonium d-10-camphorsulfonate as a standard sample. On the other hand, an aqueous solution of a d- or l-[Co(en)₃] complex can be mentioned as an example of a standard sample for observing circular dichroism intensities in the visible region. However, this solution has the problem that it undergoes photolysis when used for a long period and becomes impossible to use. Also, even if an aqueous solution of such a d- or l-[Co(en)₃] complex is used, testing and calibration needs to be performed first in the ultraviolet region, and then in the visible region, which can be troublesome and result in larger calibration errors.

Moreover, in synthesizing an optically active compound, qualitative analysis, estimation of the structure, and analysis of the state of the compound are performed using a UV-visible spectrophotometer, along with the determination of absolute configuration using a circular dichroism spectrometer.

Also in measuring the compound using a UV-visible spectrophotometer, it is necessary to test the apparatus and calibrate wavelengths, as described above.

### Non-Patent Document 1: Kiki-bunseki Guidebook (Guidebook to Instrumental Analyses), Edited by the Japan Society for Analytical Chemistry, pp. 263-266

### DISCLOSURE OF THE INVENTION

### Problem to Be Solved by the Invention

A principal object of the present invention is to provide a standard sample that allows easy and accurate testing and calibration of a circular dichroism spectrometer, and that can also be applied to the testing and calibration of a UV-visible spectrophotometer.

### Means for Solving the Problem

The present inventors conducted extensive research to solve the prior art problems, and consequently found that the aforementioned object can be achieved using a specific standard sample, thereby accomplishing the present invention.

That is to say, the present invention relates to standard samples as summarized below.

1. A standard sample for use in testing and/or calibrating a circular dichroism spectrometer and a UV-visible spectrophotometer, the standard sample comprising:

a chlorin dimer represented by Chemical Formula (I):

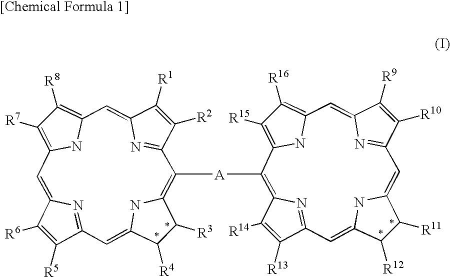

wherein A is CₙH₂ₙ (n is an integer of 0 or more); and R¹, R², R³, R⁴, R⁵, R⁶, R⁷, R⁸, R⁹R¹⁰, R¹¹, R¹², R¹³, R¹⁴, R¹⁵, and R¹⁶ are the same or different, and are each a hydrogen atom, a saturated hydrocarbon group, an aryl group that may be substituted, a heteroaryl group that may be substituted, or an aralkyl group that may be substituted; or

a metal chlorin dimer represented by Chemical Formula (II):

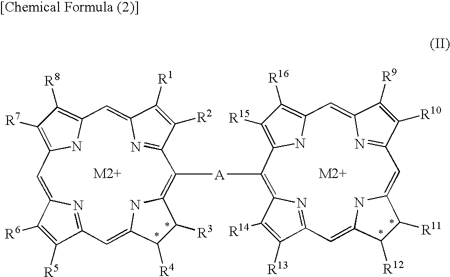

wherein M²⁺ is a divalent metal ion; and A, R¹, R², R³, R⁴, R⁵, R⁶, R⁷, R⁸, R⁹, R¹⁰, R¹¹, R¹², R¹³, R¹⁴, R¹⁵, and R¹⁶ are the same as defined above;

each of the chlorin dimer and the metal chlorin dimer exhibiting at least two peaks in an ultraviolet to visible region of a circular dichroism spectrum and a UV-visible absorption spectrum thereof.

2. The standard sample according to Item 1, wherein R¹, R², R³, R⁴, R⁵, R⁶, R⁷, R⁸, R⁹, R¹⁰, R¹¹, R¹², R¹³, R¹⁴, R¹⁵ and R¹⁶ are the same or different, and are each a C₁-C₂ linear alkyl group or a C₃-C₁₀ linear or branched alkyl group.

3. The standard sample according to Item 1, wherein the chlorin dimer is an (S,S;S,S) chlorin dimer or an (R,R;R,R) chlorin dimer, and the metal chlorin dimer is an (S,S;S,S) metal chlorin dimer or an (R,R;R,R) metal chlorin dimer.

4. The standard sample according to Item 1, wherein the chlorin dimer or the metal chlorin dimer is dissolved in a solvent.

5. The standard sample according to Item 1, wherein M²⁺ is Zn²⁺.

6. A method for producing the standard sample of Item 3, comprising:

(1) a first step of reducing a porphyrin dimer represented by Chemical Formula (III):

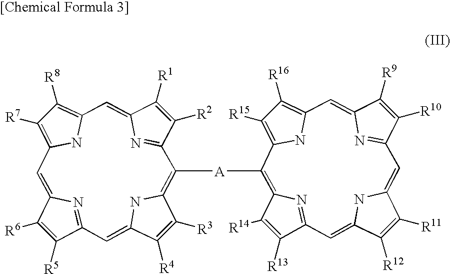

wherein A, R¹, R², R³, R⁴, R⁵, R⁶, R⁷, R⁸, R⁹, R¹⁰, R¹¹, R¹², R¹³R¹⁴, R¹⁵, and R¹⁶ are the same as defined above; to produce a racemic mixture of an (S, S/S, S) chlorin dimer and an (R, R/R, R) chlorin dimer, represented by Chemical Formula (I):

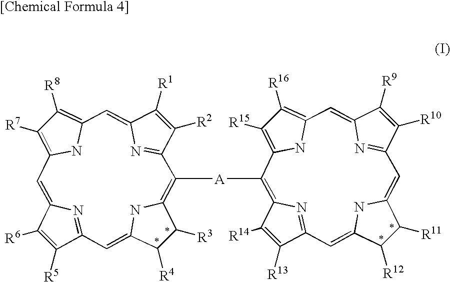

wherein A, R¹, R², R³, R⁴, R⁵, R⁶, R⁷, R⁸, R⁹, R¹⁰, R¹¹, R¹², R¹³R¹⁴, R¹⁵, and R¹⁶ are the same as defined above; and

(2) a second step of optically resolving the resulting racemic mixture to obtain an (S,S;S,S) chlorin dimer or an (R,R;R,R) chlorin dimer.

7. A method for calibrating wavelengths and circular dichroism intensities of a circular dichroism spectrometer using the standard sample of Item 1, the method comprising:

(1) a first step of preparing a chlorin dimer or a metal chlorin dimer whose circular dichroism spectrum has, in order from longer wavelengths:

(a) a wavelength X1 and an intensity Y1 of a peak showing a first Cotton effect;

(b) a wavelength X2 and an intensity Y2 of a peak showing a second Cotton effect;

(c) a wavelength X3 and an intensity Y3 of a peak showing a third Cotton effect; and

(d) a wavelength X4 and an intensity Y4 of a peak showing a fourth Cotton effect;

(2) a second step of measuring a circular dichroism spectrum of the standard sample using a circular dichroism spectrometer to be calibrated, to determine, in order from longer wavelengths:

(a) a wavelength X′1 and an intensity Y′1 of a peak showing a first Cotton effect;

(b) a wavelength X′2 and an intensity Y′2 of a peak showing a second Cotton effect;

(c) a wavelength X′3 and an intensity Y′3 of a peak showing a third Cotton effect; and

(d) a wavelength X′4 and an intensity Y′4 of a peak showing a fourth Cotton effect; and

(3) a third step of performing at least one process selected from the group consisting of: (a) calibrating the wavelength X′1 and the intensity Y′1 determined at the second step to the wavelength X1 and the intensity Y1, respectively; and (b) calibrating the wavelength X′2 and the intensity Y′2 determined at the second step to the wavelength X2 and the intensity Y2, respectively; and performing at least one process selected from the group consisting of: (c) calibrating the wavelength X′3 and the intensity Y′3 determined at the second step to the wavelength X3 and the intensity Y3, respectively; and (d) calibrating the wavelength X′4 and the intensity Y′4 to the wavelength X4 and the intensity Y4, respectively.

8. A method for calibrating wavelengths and absorbances of a UV-visible spectrophotometer using the standard sample of Item 1, the method comprising:

(1) a first step of preparing a chlorin dimer or a metal chlorin dimer whose UV-visible absorption spectrum has, in order from longer wavelengths:

(a) a wavelength X1 and an absorbance Y1 of a first peak; and

(b) a wavelength X2 and an absorbance Y2 of a second peak;

(2) a second step of measuring a UV-visible absorption spectrum of the standard sample using a UV-visible spectrophotometer to be calibrated, to determine, in order from longer wavelengths:

(a) a wavelength X′1 and an absorbance Y′1 of a first peak; and

(b) a wavelength X′2 and an absorbance Y′2 of a second peak; and

(3) a third step of calibrating the UV-visible spectrophotometer so that (a) the wavelength X′1 and the absorbance Y′1 and (b) the wavelength X′2 and the absorbance Y′2 determined at the second step become (a) the wavelength X1 and the absorbance Y1 and (b) the wavelength X2 and the absorbance Y2, respectively.

9. A method for calculating an anisotropy factor (g) of a target sample, comprising:

(1) a first step of calibrating wavelengths and circular dichroism intensities of a circular dichroism spectrometer, using the standard sample of Item 1;

(2) a second step of measuring a circular dichroism spectrum of a target sample, using the circular dichroism spectrometer calibrated in the first step, to determine circular dichroism (Δε) at the specific wavelength;

(3) a third step of calibrating wavelengths and absorbances of a UV-visible spectrophotometer, using the standard sample of Item 1;

(4) a fourth step of measuring a UV-visible absorption spectrum of the target sample, using the UV-visible spectrophotometer calibrated in the third step, to determine a molar extinction coefficient (ε) at the same wavelength as in the case of Δε; and

(5) a fifth step of dividing the circular dichroism (Δε) determined in the second step by the molar extinction coefficient (ε) determined in the fourth step to determine an anisotropy factor (g).

10. The method according to Item 9, wherein:

(1) the first step of calibrating wavelengths and circular dichroism intensities of a circular dichroism spectrometer; and (2) the second step of measuring a circular dichroism spectrum of the target sample are performed; and then successively, (3) the third step of calibrating wavelengths and absorbances of a UV-visible spectrophotometer; and (4) the fourth step of measuring a UV-visible absorption spectrum of the target sample are performed.

11. The method according to Item 9, wherein:

(1) the third step of calibrating wavelengths and absorbances of a UV-visible spectrophotometer; and (2) the fourth step of measuring a UV-visible absorption spectrum of the target sample are performed; and then successively, (3) the first step of calibrating wavelengths and circular dichroism intensities of a circular dichroism spectrometer; and (4) the second step of measuring a circular dichroism spectrum of the target sample are performed.

### Effects of the Invention

The standard sample according to the present invention exhibits two intense, sharp peaks in the ultraviolet and visible regions, and therefore allows a circular dichroism spectrometer to be tested and calibrated simultaneously at the two wavelengths. As a result, the use of the standard sample of the invention makes it possible to test and calibrate a circular dichroism spectrometer more simply and accurately than previously.

Also, the standard sample of the invention can be used to test and/or calibrate a UV-visible spectrophotometer. This allows a circular dichroism spectrometer and a UV-visible spectrophotometer to be tested and/or calibrated with a single standard sample, to determine the absolute configuration of, and perform qualitative and quantitative analyses of, an optically active substance, more simply and accurately than previously.

Moreover, anisotropy factors g (g=Δε/ε) of an optically active compound can be accurately calculated by using a single standard sample, and testing and/or calibrating a circular dichroism spectrometer and a UV-visible spectrophotometer simultaneously at two wavelengths.

### BRIEF DESCRIPTION OF THE DRAWINGS

FIG. 1 shows a circular dichroism spectrum and a UV-visible absorption spectrum for each of the (S,S;S,S) and (R,R;R,R) enantiomers of Chlorin (IV).

### BEST MODE FOR CARRYING OUT THE INVENTION

### Standard Sample

The standard sample of the present invention is a standard sample for use in testing and/or calibrating a circular dichroism spectrometer and a UV-visible spectrophotometer. The standard sample comprises a chlorin dimer represented by Chemical Formula (I):

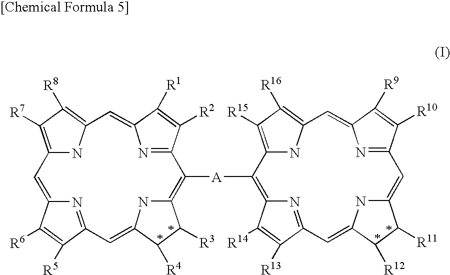

wherein A is CₙH₂ₙ (n is an integer of 0 or more); and R¹, R², R³, R⁴, R⁵, R⁶, R⁷, R⁸, R⁹, R¹⁰, R¹¹, R¹², R¹³, R¹⁴, R¹⁵, and R¹⁶ are the same or different, and are each a hydrogen atom, a saturated hydrocarbon group, an aryl group that may be substituted, a heteroaryl group that may be substituted, or an aralkyl group that may be substituted; or

a metal chlorin dimer represented by Chemical Formula (II):

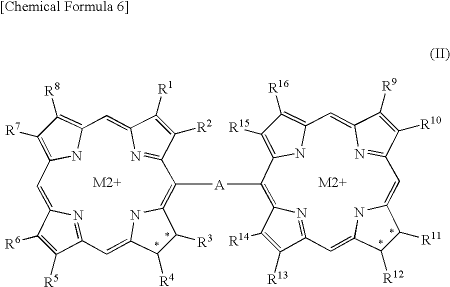

wherein M²⁺ is a divalent metal ion; and A, R¹, R², R³, R⁴, R⁵, R⁶, R⁷, R⁸, R⁹, R¹⁰, R¹¹, R¹², R¹³, R¹⁴, R¹⁵, and R¹⁶ are the same as defined above.

Each of the chlorin dimer and the metal chlorin dimer exhibits at least two peaks in an ultraviolet to visible region (specifically, in the range of wavelengths at 300 to 700 nm) of a circular dichroism spectrum and a UV-visible absorption spectrum thereof.

The standard sample is not particularly limited as long as it comprises the chlorin dimer or metal chlorin dimer, but is preferably such that the chlorin dimer or metal chlorin dimer is dissolved in a solvent. When the chlorin dimer or metal chlorin dimer is dissolved in a solvent, the standard sample is easy to handle. Also, when the chlorin dimer or metal chlorin dimer is dissolved in a solvent and sealed in optical cells for measurement, they can be stored for a long period.

Although the solvent is not particularly limited, it is preferably a solvent that does not have an absorption at 300 nm or more. Examples of such solvents include dichloromethane, chloroform, dichloroethane, pentane, hexane, heptane and the like. Among these examples, dichloromethane and chloroform are particularly preferable.

The group represented by A is not particularly limited as long as it is CₙH₂ₙ (wherein n is an integer of 0 or more). Preferably, n in the CₙH₂ₙ represented by A is an integer from 0 to 4, and more preferably, an integer of 2 (i.e., C₂H₄).

Saturated hydrocarbon groups are not particularly limited, and examples include C₁-C₁₀ linear or branched alkyl groups, C₃-C₈ cycloalkyl groups, and the like. Examples of C₁-C₁₀ linear or branched alkyl groups include methyl, ethyl, propyl, iso-propyl, tert-butyl groups and the like. Examples of C₃-C₈ cycloalkyl groups include cyclopropyl, cyclobutyl groups and the like.

The aryl group that may be substituted is not particularly limited, and may, for example, be C₆-C₁₄ aryl. Specific examples include phenyl, 1-naphthyl, 2-naphthyl, biphenylyl, anthryl and the like. Examples of substituents of the aryl group that may be substituted include C₁-C₆ alkyl, C₆-C₁₄ aryl, 5- to 10-membered aromatic heterocyclic groups, and the like. Examples of C₁-C₆ alkyl include methyl, ethyl, propyl, isopropyl, butyl, isobutyl, sec-butyl, tert-butyl, pentyl, hexyl and the like. Examples of C₆-C₁₄ aryl include phenyl, 1-naphthyl, 2-naphthyl, biphenylyl, 2-anthryl and the like. Examples of 5- to 10-membered aromatic heterocyclic groups include 2- or 3-thienyl; 2-, 3-, or 4-pyridyl; 2-, 3-, 4-, 5-, or 8-quinolyl; 1-, 3-, 4-, or 5-isoquinolyl; 1-, 2-, or 3-indolyl; 2-benzothiazolyl; 2-benzo[b]thienyl; benzo[b]furanyl; and the like.

Substituents of the aryl group that may be substituted are not particularly limited, but may, for example, be alkyl groups and amino groups. The position at which the aryl group is substituted with a substituent is not particularly limited, and may be suitably determined according to the purpose. The number of substituents is not particularly limited, but is preferably 1 to 3, and more preferably 1 or 2.

The heteroaryl group of the heteroaryl group that may be substituted refers to a 5- to 14-membered aromatic heterocyclic group that contains one to three atoms of sulfur, oxygen, and/or nitrogen and may form a fused ring; examples include furyl, thienyl, pyrrolyl, pyrazolyl, imidazolyl, oxazolyl, isoxazolyl, isothiazolyl, thiazolyl, 1,2,3-oxadiazolyl, triazolyl, tetrazolyl, thiadiazolyl, pyridyl, pyridazinyl, pyrimidinyl, pyrazinyl, indolyl, indazolyl, purinyl, quinolyl, isoquinolyl, phthalazinyl, naphthyridinyl, quinoxalinyl, quinazolinyl, cinnolinyl, pteridinyl, carbazolyl, caribolinyl, phenanthridinyl, and acridinyl. The types of substituents of the heteroaryl group that may be substituted, as well as the positions and the number of the substituents, are the same as described above with respect to the aryl group that may be substituted.

Examples of the aralkyl group that may be substituted include benzyl, phenethyl, and naphthyl groups. The types of substituents of the aralkyl group that may be substituted, as well as the positions and the number of the substituents, are the same as described above with respect to the aryl group that may be substituted.

In the present invention, in particular, R¹, R², R³, R⁴, R⁵, R⁶, R⁷, R⁸, R⁹, R¹⁰, R¹¹, R¹², R¹³, R¹⁴, R¹⁵ and R¹⁶ are preferably the same or different, and are each a C₁-C₂ linear alkyl group or a C₃-C₁₀ linear or branched alkyl group; and more preferably, the R groups are all ethyl groups.

Although the chlorin dimer is not particularly limited, it is preferably an (S,S;S,S) chlorin dimer or an (R,R;R,R) chlorin dimer.

Although the metal chlorin dimer is not particularly limited, it is preferably an (S,S;S,S) metal chlorin dimer or an (R,R;R,R) metal chlorin dimer.

M²⁺ in the aforementioned compound (2) is not particularly limited as long as it is a divalent metal ion. Examples include Zn²⁺, Mg²⁺, Co²⁺, Ni²⁺, Cu²⁺, Pd²⁺, Pt²⁺ and the like. Among these examples, Zn²⁺ is particularly preferable.

Each of the chlorin dimer and the metal chlorin dimer exhibits at least two peaks in an ultraviolet to visible region of a circular dichroism spectrum or a UV-visible absorption spectrum thereof. For example, when a circular dichroism spectrum is measured for a standard sample comprising an (S,S;S,S) enantiomer of the dizinc chlorin dimer (hereinafter abbreviated to “Chlorin (IV)”), represented by Chemical Formula (IV):

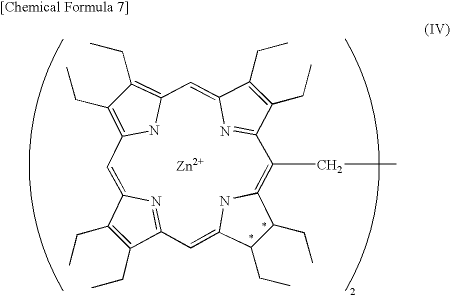

the (S,S;S,S) enantiomer of Chlorin (IV) exhibits (a) a peak showing a first Cotton effect (wavelength: 633 mm, intensity: +76 cm⁻¹ M⁻¹); (b) a peak showing a second Cotton effect (wavelength: 618 nm, intensity: −55 cm⁻¹ M⁻¹); (c) a peak showing a third Cotton effect (wavelength: 416 nm, intensity: −92 cm⁻¹ M⁻¹); and (d) a peak showing a fourth Cotton effect (wavelength: 402 nm, intensity: +71 cm⁻¹ M⁻¹).

Also, when a UV-visible absorption spectrum of the (S,S;S,S) enantiomer of Chlorin (IV) is measured, the (S,S;S,S) enantiomer of Chlorin (IV) exhibits a first peak (wavelength: 623 nm, absorbance: 0.249 and molar extinction coefficient (ε): 67000 cm⁻¹ M⁻¹) and a second peak (wavelength: 406 nm, absorbance: 0.700 and ε: 190000 cm⁻¹ M⁻¹).

The use of this standard sample allows testing and calibration to be performed with high accuracy.

### Method for Producing the Standard Sample

The standard sample of the present invention can be produced through the specific steps of a reduction process and optical resolution.

As representative examples, 1) a method for producing a standard sample comprising an (S,S;S,S) chlorin dimer or an (R,R;R,R) chlorin dimer, and 2) a method for producing an (S,S;S,S) metal chlorin dimer or an (R,R;R,R) metal chlorin dimer will be described in detail below.

The method for producing a standard sample comprising an (S,S;S,S) chlorin dimer or an (R,R;R,R) chlorin dimer includes

(1) a first step of reducing a porphyrin dimer represented by Chemical Formula (III) (hereinafter abbreviated to “Chlorin (III)”):

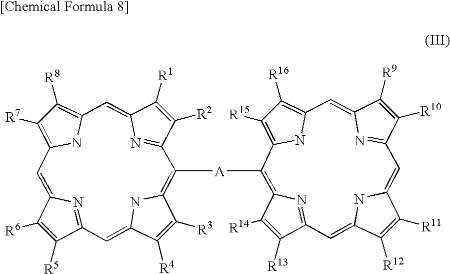

wherein A, R¹, R², R³, R⁴, R⁵, R⁶, R⁷, R⁸, R⁹, R¹⁰, R¹¹, R¹², R¹³R¹⁴, R¹⁵, and R¹⁶ are the same as defined above; to produce a racemic mixture of an (S, S/S, S) chlorin dimer and an (R, R/R, R) chlorin dimer, represented by Chemical Formula (I):

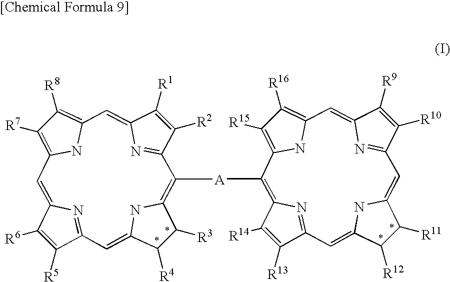

wherein A, R¹, R², R³, R⁴, R⁵, R⁶, R⁷, R⁸, R⁹, R¹⁰, R¹¹, R¹², R¹³R¹⁴, R¹⁵, and R¹⁶ are the same as defined above; and

(2) a second step of optically resolving the resulting racemic mixture to obtain an (S,S;S,S) chlorin dimer or an (R,R;R,R) chlorin dimer.

### First Step

In the first step, the aforementioned Chlorin (III) is reduced to produce the racemic mixture of an (S,S;S,S) chlorin dimer and an (R,R;R,R) chlorin dimer, represented by Chemical Formula (I) shown above.

Chlorin (III) is a known compound that is readily available according to the standard procedure. The racemic mixture can also be readily produced according to a known method. For example, the racemic mixture represented by Chemical Formula (V):

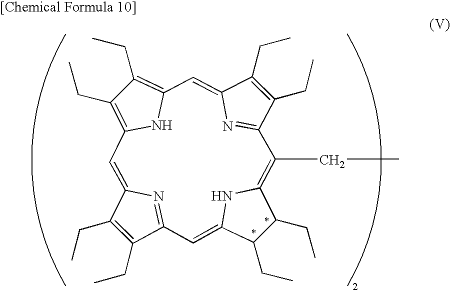

can be produced by allowing copper octaethylchlorin to react with a Vilsmeier complex POCl₃-DMF, reducing the resulting Vilsmeier salt with NaBH₄, and refluxing the reduction product in dichloromethane in the presence of methyl iodide.

Reduction may be performed according to a known reduction method. For example, a reduction method as described in “K. M. Smith, Porphyrins and Metalloporphyrins, Elsevier, Amsterdam, 1975. p. 815” may be mentioned.

After reduction, the reaction mixture may be purified to yield a racemic mixture of an (S,S;S,S) chlorin dimer and an (R,R;R,R) chlorin dimer, according to common purification procedures such as column chromatography, re-crystallization, and the like.

The absolute configuration may be determined based on the sign of the Cotton effect of a measured circular dichroism spectrum.

### Second Step

In the second step, the resulting racemic mixture is optically resolved to yield an (S,S;S,S) chlorin dimer or an (R,R;R,R) chlorin dimer.

The optical resolution of the racemic mixture of an (S,S;S,S) chlorin dimer and an (R,R;R,R) chlorin dimer may be performed using, for example, high-performance liquid chromatography (HPLC). Although the column used in optical resolution using HPLC is not particularly limited, it is preferably the tradename “Chiralcel® OJ-RH” (manufactured by Daicel Chemical Industries, Ltd.). Although the developing solvent is not particularly limited, it is preferably methanol. The flow rate may be suitably set according to the developing solvent used and the like, but is preferably 1 to 10 ml/min.

The resulting (S,S;S,S) chlorin dimer or (R,R;R,R) chlorin dimer may be dissolved in any of the solvents mentioned above.

The method for producing a standard sample comprising an (S,S;S,S) metal chlorin dimer or an (R,R;R,R) metal chlorin dimer may include the steps of i) producing a racemic mixture of an (S,S;S,S) chlorin dimer and an (R,R;R,R) chlorin dimer; and ii) optically resolving the resulting racemic mixture to obtain an (S,S;S,S) metal chlorin dimer or an (R,R;R,R) metal chlorin dimer.

The method for producing a racemic mixture of an (S,S;S,S) metal chlorin dimer and an (R,R;R,R) metal chlorin dimer is not particularly limited; a racemic mixture of an (S,S;S,S) metal chlorin dimer and an (R,R;R,R) metal chlorin dimer may be prepared using the method explained above, and then a metal may be inserted into the (S,S;S,S) chlorin dimer and the (R,R;R,R) chlorin dimer according to a known method.

The method for optically resolving the racemic mixture of an (S,S;S,S) metal chlorin dimer and an (R,R;R,R) metal chlorin dimer is not particularly limited, and may be the same as that described in the second step.

### Method for Calibrating Wavelengths and Circular Dichroism

### Intensities of Circular Dichroism Spectrometer

The present invention further includes the calibration method described below. The calibration method of the invention is a method for calibrating wavelengths and circular dichroism intensities of a circular dichroism spectrometer using the above-described standard sample. The method includes (1) a first step of preparing a chlorin dimer or a metal chlorin dimer whose circular dichroism spectrum has, in order from longer wavelengths:

(a) a wavelength X1 and an intensity Y1 of a peak showing a first Cotton effect;
(b) a wavelength X2 and an intensity Y2 of a peak showing a second Cotton effect;
(c) a wavelength X3 and an intensity Y3 of a peak showing a third Cotton effect; and
(d) a wavelength X4 and an intensity Y4 of a peak showing a fourth Cotton effect; and
(2) a second step of measuring a circular dichroism spectrum of the standard sample, using a circular dichroism spectrometer to be calibrated, to determine, in order from longer wavelengths:
(a) a wavelength X′1 and an intensity Y′1 of a peak showing a first Cotton effect;
(b) a wavelength X′2 and an intensity Y′2 of a peak showing a second Cotton effect;
(c) a wavelength X′3 and an intensity Y′3 of a peak showing a third Cotton effect; and
(d) a wavelength X′4 and an intensity Y′4 of a peak showing a fourth Cotton effect; and
(3) a third step of performing at least one process selected from the group consisting of: (a) calibrating the wavelength X′1 and the intensity Y′1 determined at the second step to the wavelength X1 and the intensity Y1, respectively; and (b) calibrating the wavelength X′2 and the intensity Y′2 determined at the second step to the wavelength X2 and the intensity Y2, respectively; and performing at least one process selected from the group consisting of: (c) calibrating the wavelength X′3 and the intensity Y′3 determined at the second step to the wavelength X3 and the intensity Y3, respectively; and (d) calibrating the wavelength X′4 and the intensity Y′4 to the wavelength X4 and the intensity Y4, respectively.

### First Step

In the first step, a compound is prepared, as the aforementioned compound, whose circular dichroism spectrum has, in order from longer wavelengths: (a) a wavelength X1 and an intensity Y1 of a peak showing a first Cotton effect; (b) a wavelength X2 and an intensity Y2 of a peak showing a second Cotton effect; (c) a wavelength X3 and an intensity Y3 of a peak showing a third Cotton effect; and (d) a wavelength X4 and an intensity Y4 of a peak showing a fourth Cotton effect.

For example, an (S,S;S,S) enantiomer of Chlorin (IV) may be prepared that has: (a) a wavelength of 633 nm and an intensity of +76 cm⁻¹ M⁻¹ of a peak showing a first Cotton effect; (b) a wavelength of 618 nm and an intensity of −55 cm⁻¹ M⁻¹ of a peak showing a second Cotton effect; (c) a wavelength of 416 nm and an intensity of −92 cm⁻¹ M⁻¹ of a peak showing a third Cotton effect; and (d) a wavelength of 402 nm and an intensity of +71 cm⁻¹ M⁻¹ of a peak showing a fourth Cotton effect.

### Second Step

In the second step, a circular dichroism spectrum of the standard sample is measured, using a circular dichroism spectrometer to be calibrated, to determine, in order from longer wavelengths: (a) a wavelength X′1 and an intensity Y′1 of a peak showing a first Cotton effect; (b) a wavelength X′2 and an intensity Y′2 of a peak showing a second Cotton effect; (c) a wavelength X′3 and an intensity Y′3 of a peak showing a third Cotton effect; and (d) a wavelength X′4 and an intensity Y′4 of a peak showing a fourth Cotton effect.

The circular dichroism spectrometer to be calibrated is not particularly limited, and may be a known or commercially available circular dichroism spectrometer.

The circular dichroism spectrum may be measured according to a known method that is suitable for the circular dichroism spectrometer used.

The wavelengths X′1, X′2, X′3, and X′4 and the intensities Y′1, Y′2, Y′3, and Y′4 are not necessarily constant numerical values, and vary depending on the condition and the like of the circular dichroism spectrometer used. That is to say, the accuracy of the circular dichroism spectrometer is reduced by this variation. For this reason, calibration is performed at the third step using the standard sample.

### Third Step

In the third step, at least one process is performed selected from the group consisting of: (a) calibrating the wavelength X′1 and the intensity Y′1 determined at the second step to the wavelength X1 and the intensity Y1, respectively; and (b) calibrating the wavelength X′2 and the intensity Y′2 determined at the second step to the wavelength X2 and the intensity Y2, respectively; and performing at least one process selected from the group consisting of: (c) calibrating the wavelength X′3 and the intensity Y′3 determined at the second step to the wavelength X3 and the intensity Y3, respectively; and (d) calibrating the wavelength X′4 and the intensity Y′4 to the wavelength X4 and the intensity Y4, respectively.

That is to say, the apparatus is corrected so that the wavelengths X′1, etc., and the intensities Y′1, etc., determined at the second step become the original wavelengths X1, etc., and the original intensities Y1, etc, respectively. Calibration may be performed using a suitable method according to the specification and the like of an apparatus to be calibrated.

### Method for Calibrating Wavelengths and Absorbances of a UV-Visible Spectrophotometer

The present invention further includes the calibration method described below. The calibration method of the invention is a method for calibrating wavelengths and absorbances of a UV-visible spectrophotometer using the standard sample. The method includes:

(1) a first step of preparing a chlorin dimer or a metal chlorin dimer whose UV-visible absorption spectrum has, in order from longer wavelengths:
(a) a wavelength X1 and an absorbance Y1 of a first peak, and
(b) a wavelength X2 and an absorbance Y2 of a second peak;
(2) a second step of measuring a UV-visible absorption spectrum of the standard sample, using a UV-visible spectrophotometer to be calibrated, to determine, in order from longer wavelengths:
(a) a wavelength X′1 and an absorbance Y′1 of a first peak, and
(b) a wavelength X′2 and an absorbance Y′2 of a second peak; and
(3) a third step of calibrating the UV-visible spectrophotometer so that (a) the wavelength X′1 and the absorbance Y′1 and (b) the wavelength X′2 and the absorbance Y′2 determined at the second step become (a) the wavelength X1 and the absorbance Y1 and (b) the wavelength X2 and the absorbance Y2, respectively.

### First Step

In the first step, a compound is prepared, as the aforementioned compound, whose UV-visible absorption spectrum has, in order from longer wavelengths: (a) a wavelength X1 and an absorbance Y1 of a first peak; and (b) a wavelength X2 and an absorbance Y2 of a second peak.

For example, an (S,S;S,S) enantiomer of Chlorin (IV) may be prepared that has a wavelength of 623 nm and an absorbance of 0.249 (a molar extinction coefficient (ε) of 67000 cm⁻¹ M⁻¹) for the first peak; and (b) a wavelength of 406 nm and an absorbance of 0.700 (ε of 190000 cm⁻¹ M⁻¹) for the second peak.

### Second Step

In the second step, a UV-visible absorption spectrum of the standard sample is measured, using a UV-visible spectrophotometer to be calibrated, to determine, in order from longer wavelengths:

(a) a wavelength X′1 and an absorbance Y′1 of a first peak; and
(b) a wavelength X′2 and an absorbance Y′2 of a second peak.

The UV-visible absorption spectrum may be measured according to a known method that is suitable for the type of the UV-visible spectrophotometer used.

The wavelengths X′1, X′2 and the intensities Y′1, Y′2 are not necessarily constant numerical values, and vary depending on the condition and the like of the UV-visible spectrophotometer used. That is to say, the accuracy of the UV-visible spectrophotometer is reduced by this variation. For this reason, calibration is performed at the third step using the standard sample.

### Third Step

In the third step, the UV-visible spectrophotometer is calibrated so that (a) the wavelength X′1 and the absorbance Y′1; and (b) the wavelength X′2 and the absorbance Y′2 determined at the second step become (a) the wavelength X1 and the absorbance Y1; and (b) the wavelength X2 and the absorbance Y2, respectively.

That is to say, the wavelengths X′1, etc., and the intensities Y′1, etc., determined at the second step are calibrated to the original wavelengths X1, etc., and the original intensities Y1, etc., respectively. Calibration may be performed according to the procedures suitable for the specification of each apparatus.

The calibration method of the present invention can also be applied to the calibration of wavelengths and absorbances of a UV-visible spectroscope attached to a circular dichroism spectrometer, in addition to a UV-visible spectrophotometer.

### Method for Calculating Anisotropy Factor

The method for calculating an anisotropy factor (g) of the present invention is a method for calculating an anisotropy factor (g) of a target sample, which includes:

(1) a first step of calibrating wavelengths and circular dichroism intensities of a circular dichroism spectrometer using the standard sample as defined above;
(2) a second step of measuring a circular dichroism spectrum of a target sample, using the circular dichroism spectrometer calibrated in the first step, to determine circular dichroism (Δε) at the specific wavelength;
(3) a third step of calibrating wavelengths and absorbances of a UV-visible spectrophotometer using the standard sample;
(4) a fourth step of measuring a UV-visible spectrum of the target sample, using the UV-visible spectrophotometer calibrated in the third step, to determine a molar extinction coefficient (ε) at the specific wavelength (the same as in the case of Δε); and
(5) a fifth step of dividing the circular dichroism (Δε) determined in the second step by the molar extinction coefficient
(ε) determined in the fourth step, to determine an anisotropy factor (g) at the specific wavelength.

### First Step

In the first step, wavelengths and circular dichroism intensities of a circular dichroism spectrometer are calibrated using the standard sample as defined above.

The circular dichroism spectrometer is not particularly limited, and a commercially available circular dichroism spectrometer may be used.

This calibration may be performed in the same manner as described in the section “Method for Calibrating Wavelengths and Circular Dichroism Intensities of a Circular dichroism spectrometer” shown above.

### Second Step

In the second step, circular dichroism (Δε) is determined by measuring a circular dichroism spectrum of the target sample, using the circular dichroism spectrometer calibrated in the first step.

That is to say, a circular dichroism spectrum of the target sample is measured using the circular dichroism spectrometer calibrated in the first step. This enables accurate measurement of a circular dichroism spectrum of the target sample. As a result, the circular dichroism (Δε) can be determined with high accuracy.

The target sample is not particularly limited as long as it does not impair the effects attained by the present invention.

### Third Step

In the third step, wavelengths and absorbances of a UV-visible spectrophotometer are calibrated using the standard sample.

The method of the invention can be applied to any known UV-visible spectrophotometer.

Calibration may be performed in the same manner as described in the section “Method for Calibrating Wavelengths and Absorbances of a UV-Visible Spectrophotometer”.

### Fourth Step

In the fourth step, a UV-visible absorption spectrum of the target sample is measured, using the UV-visible spectrophotometer calibrated in the third step, to determine a molar extinction coefficient (s).

That is to say, a UV-visible absorption spectrum of the target sample is measured using the UV-visible spectrophotometer calibrated in the third step. This enables accurate measurement of a UV-visible absorption spectrum of the target sample. As a result, the molar extinction coefficient (s) can be determined with high accuracy.

### Fifth Step

In the fifth step, an anisotropy factor (g) is calculated by dividing the circular dichroism (Δε) determined at the specific wavelength(s) in the second step by the molar extinction coefficient (e) determined at the specific wavelength(s) (the same as in the case of Δε) in the fourth step.

The term “anisotropy factor (g)” as used herein is a value inherent in optically active compounds, and is an important factor that affects the optical yield of an absolute asymmetric synthesis reaction by irradiation of circularly polarized light. When the anisotropy factor (g) is high, a circular dichroism spectrum of even a sample with low optical purity can be measured. Also, the efficiency of absolute asymmetric synthesis using circularly polarized light increases to thereby improve the optical yield.

In particular, in the method for calculating an anisotropy factor (g) of the present invention, it is preferable that (1) the first step of calibrating wavelengths and circular dichroism intensities of a circular dichroism spectrometer; and (2) the second step of measuring a circular dichroism spectrum of the target sample are performed; and then successively, (3) the third step of calibrating wavelengths and absorbances of a UV-visible spectrophotometer; and (4) the fourth step of measuring a UV-visible absorption spectrum of the target sample are performed. That is to say, because the standard sample of the invention can be used for calibrating a circular dichroism spectrometer and a UV-visible spectrophotometer, the third step and the fourth step can be performed successively after the first step and the second step. This enables an anisotropy factor (g) to be calculated with higher accuracy.

In the method for calculating an anisotropy factor (g) of the invention, the third step and the fourth step may precede the first step and the second step.

That is to say, according to the method for calculating an anisotropy factor (g) of the present invention, an anisotropy factor (g) even with higher accuracy can be achieved when (1) the third step of calibrating wavelengths and absorbances of a UV-visible spectrophotometer; and (2) the fourth step of measuring a UV-visible absorption spectrum of the target sample are performed; and then successively, (3) the first step of calibrating wavelengths and circular dichroism intensities of a circular dichroism spectrometer; and (4) the second step of measuring a circular dichroism spectrum of the target sample are performed.

### EXAMPLES

The present invention will be described in detail below with reference to Preparation Examples, Examples, and Comparative Examples; the invention is not limited by these Examples and the like.

As a circular dichroism spectrometer, a product marketed under the tradename “J-820” (manufactured by JASCO Corp.) was used.

As a UV-visible spectrophotometer, a product marketed under the tradename “V-550” (manufactured by JASCO Corp.) was used.

### Preparation Example 1

An (S,S;S,S) enantiomer and an (R,R;R,R) enantiomer of Chlorin (IV) were produced in the following manner.

One gram of Fe (III) octaethylporphyrin, 10 g of sodium, and 100 ml of iso-amyl alcohol were placed in a reactor and heated under reflux for 30 minutes. The reaction mixture was then stirred in a solution mixture of 500 ml of acetic acid containing iron (II) sulfate and 150 ml of concentrated hydrochloric acid to yield octaethylchlorin.

Then, 250 mg of octaethylchlorin, an excess of Cu (OAc), and 100 ml of a solution mixture of CHCl₃ and MeOH(CHCl₃:MeOH=100:1) were placed in a reactor and heated under reflux for 2 to 3 minutes to yield copper octaethylchlorin.

After this, 710 mg of the copper octaethylchlorin, 26 ml of POCl₃, 22 ml of dimethylformamide (DMF), and 500 ml of CH₂ClCH₂Cl were placed in a reactor and stirred at 60° C. for 45 minutes. The resulting stirred solution was reduced in 500 ml of a solution mixture of CHCl₃ and MeOH(CHCl₃: MeOH=3:1) at room temperature (25° C.), using 1.2 g of NaBH₄. To the reactor containing the resulting reduction product was added 35 ml of MeI and 140 ml of CH₂Cl₂, and the reaction mixture was stirred while heating for 3 hours. A 90 ml portion of concentrated sulfuric acid was then added to the resulting product, and the mixture was stirred at room temperature (25° C.) for 3.5 hours to yield an octaethylchlorin dimer.

Then, 250 mg of the octaethylchlorin dimer, an excess of Zn (OAc), and 100 ml of a solution mixture of CHCl₃ and MeOH(CHCl₃:MeOH=100:1) were placed in a reactor and heated under reflux for 2 to 3 minutes to yield a racemic mixture of an (S,S;S,S) enantiomer and an (R,R;R,R) enantiomer of Chlorin (IV).

The resulting mixture was optically resolved using HPLC, thereby yielding an (S,S;S,S) enantiomer and an (R,R;R,R) enantiomer. As a column attached to the HPLC, the product name “Chiralcel® OJ-RH column” (manufactured by Daicel Chemical Industries, Ltd.) was used. Methanol was used as a developing solvent. The flow rate was 5.5 ml/min.

3.7×10⁻⁸ mol of the prepared (S,S;S,S) enantiomer was dissolved in 10 ml of dichloromethane to prepare Standard Sample 1.

Also, 3.7×10⁻⁸ mol of the (R,R;R,R) enantiomer was dissolved in 10 ml of dichloromethane to prepare Standard Sample 2.

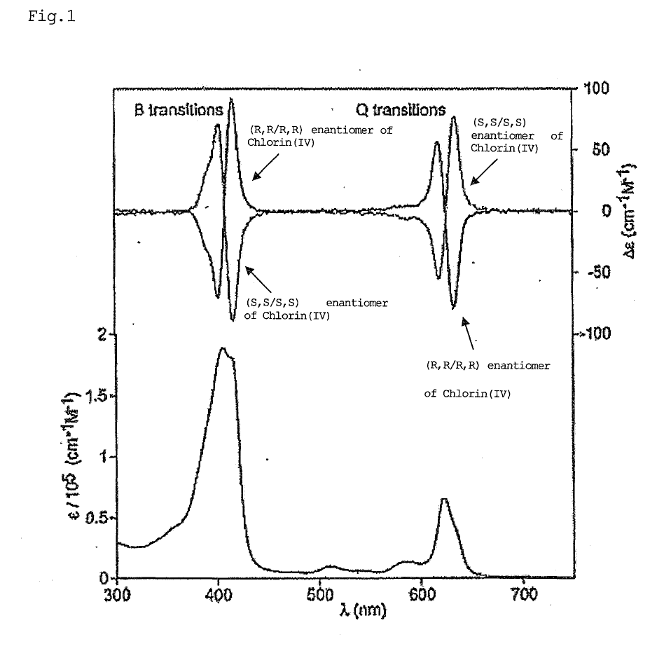

When a circular dichroism spectrum of the (S,S;S,S) enantiomer was measured, the measured spectrum had, in order from longer wavelengths: (a) a peak showing a first Cotton effect (wavelength: 633 nm, intensity: +76 cm⁻¹ M⁻¹); (b) a peak showing a second Cotton effect (wavelength: 618 nm, intensity: −55 cm⁻¹ M⁻¹); (c) a peak showing a third Cotton effect (wavelength: 416 nm, intensity: −92 cm⁻¹ M⁻¹); and (d) a peak showing a fourth Cotton effect (wavelength: 402 nm, intensity: +71 cm¹ M⁻¹). FIG. 1 shows the measured circular dichroism spectrum.

Also, when a UV-visible absorption spectrum of the (S,S;S,S) enantiomer was measured, the measured spectrum had, in order from longer wavelengths, a first peak (wavelength: 623 nm, absorbance: 0.249 and E: 67000 cm⁻¹ M⁻¹) and a second peak (wavelength: 406 nm, absorbance: 0.700 and ε: 190000 cm⁻¹ M⁻¹). FIG. 1 shows the measured UV-visible absorption spectrum.

When a circular dichroism spectrum of the (R,R;R,R) enantiomer was measured, the measured spectrum had, in order from longer wavelengths: (a) a peak showing a first Cotton effect (wavelength: 633 nm, intensity: −76 cm⁻¹ M⁻¹); (b) a peak showing a second Cotton effect (wavelength: 618 nm, intensity: +55 cm⁻¹ M⁻¹); (c) a peak showing a third Cotton effect (wavelength: 416 nm, intensity: +92 cm⁻¹ M⁻¹); and (d) a peak showing a fourth Cotton effect (wavelength: 402 nm, intensity: −71 cm⁻¹ M⁻¹). FIG. 1 shows the measured circular dichroism spectrum.

Also, when a UV-visible absorption spectrum of the (R,R;R,R) enantiomer was measured, the measured spectrum had, in order from longer wavelengths, a first peak (wavelength: 623 nm, absorbance: 0.249 and ε: 67000 cm⁻¹ M⁻¹) and a second peak (wavelength: 406 nm, absorbance: 0.700 and ε: 190000 cm⁻¹ M⁻¹). FIG. 1 shows the measured UV-visible absorption spectrum.

### Example 1

By measuring a circular dichroism spectrum of Standard Sample 1, the following peaks were determined in order from longer wavelengths: (a) a peak showing a first Cotton effect (wavelength: 636 nm, intensity: +72 cm⁻¹ M⁻¹); (b) a peak showing a second Cotton effect (wavelength: 621 nm, intensity: −52 cm⁻¹ M⁻¹); (c) a peak showing a third Cotton effect (wavelength: 418 nm, intensity: −88 cm⁻¹ M⁻¹); and (d) a peak showing a fourth Cotton effect (wavelength: 404 nm, intensity: +68 cm⁻¹ M⁻¹).

The circular dichroism spectrometer was calibrated so that the determined wavelength of 636 nm and intensity of +72 cm⁻¹ M⁻¹ of the peak showing the first Cotton effect became a wavelength of 633 nm and an intensity of +76 cm⁻¹ M⁻¹, respectively; the wavelength of 621 nm and intensity of −52 cm⁻¹ M⁻¹ of the peak showing the second Cotton effect became a wavelength of 618 nm and an intensity of −55 cm⁻¹ M⁻¹, respectively; the wavelength of 418 nm and intensity of −88 cm⁻¹ M⁻¹ of the peak showing the third Cotton effect became a wavelength of 416 nm and an intensity of −92 cm⁻¹ M⁻¹, respectively; and the wavelength of 404 nm and intensity of +68 cm⁻¹ M⁻¹ of the peak showing the fourth Cotton effect became a wavelength of 402 nm and an intensity of +71 cm⁻¹ M⁻¹, respectively.

The wavelengths of the circular dichroism spectrometer were calibrated by turning the coarse adjustment screw and the fine adjustment screw of the wavelength lever.

The circular dichroism intensities of the circular dichroism spectrometer were calibrated, subsequent to the calibration of wavelengths, by turning the “Scale Correction CD” trimmer on the rear panel of the amplifier of the circular dichroism spectrometer.

### Example 2

The measurement of the UV-visible absorption spectrum of Standard Sample 1 confirmed that Standard Sample 1 exhibits a first peak (wavelength: 626 nm, absorbance: 0.255) and a second peak (wavelength: 408 nm, absorbance: 0.705) in order from longer wavelengths.

The UV-visible spectrophotometer was calibrated so that the determined wavelength of 626 nm and absorbance of 0.255 of the first peak became a wavelength of 623 nm and an absorbance of 0.249, respectively, and the wavelength of 408 nm and absorbance of 0.705 of the second peak became a wavelength of 406 nm and an absorbance 0.700, respectively.

The wavelengths of the UV-visible spectrophotometer were calibrated using the wavelength calibration function of the UV-visible spectrophotometer.

The absorbances of the UV-visible spectrophotometer were calibrated so that they corresponded to the known absorbances of the first and second peaks by turning the coarse adjustment screw and the fine adjustment screw to adjust the intensity axis.

### Example 3

By measuring a circular dichroism spectrum of Standard Sample 2, the following peaks were determined in order from longer wavelengths: (a) a peak showing a first Cotton effect (wavelength: 636 nm, intensity: −72 cm⁻¹ M⁻¹); (b) a peak showing a second Cotton effect (wavelength: 621 nm, intensity: +52 cm⁻¹ M⁻¹); (c) a peak showing a third Cotton effect (wavelength: 418 nm, intensity: +88 cm⁻¹ M⁻¹); and (d) a peak showing a fourth Cotton effect (wavelength: 404 nm, intensity: −68 cm⁻¹ M⁻¹).

The circular dichroism spectrometer was calibrated so that the determined wavelength of 636 nm and intensity of −72 cm⁻¹ M⁻¹ of the peak showing the first Cotton effect became a wavelength of 633 nm and an intensity of −76 cm⁻¹ M⁻¹, respectively; the wavelength of 621 nm and intensity of +52 cm⁻¹ M⁻¹ of the peak showing the second Cotton effect became a wavelength of 618 nm and an intensity of +55 cm⁻¹ M⁻¹, respectively; the wavelength of 418 nm and intensity of +88 cm⁻¹ M⁻¹ of the peak showing the third Cotton effect became a wavelength of 416 nm and an intensity of +92 cm⁻¹ M⁻¹, respectively; and the wavelength of 404 nm and intensity of −68 cm⁻¹ M⁻¹ of the peak showing the fourth Cotton effect became a wavelength of 402 nm and an intensity of −71 cm⁻¹ M⁻¹, respectively.

The wavelengths and intensities of the circular dichroism spectrometer were calibrated in the same manner as described in Example 1.

### Example 4

The measurement of a UV-visible spectrum of Standard Sample 2 confirmed that Standard Sample 2 exhibits a first peak (wavelength: 626 nm, absorbance: 0.255) and a second peak (wavelength: 408 nm, absorbance: 0.705) in order from longer wavelengths.

The UV-visible spectrophotometer was calibrated so that the determined wavelength of 626 nm and absorbance of 0.255 of the first peak became a wavelength of 623 nm and an absorbance of 0.249, respectively, and the wavelength of 408 nm and absorbance of 0.705 of the second peak became a wavelength of 406 nm and an absorbance 0.700, respectively.

The wavelengths and absorbances of the UV-visible spectrophotometer were calibrated in the same manner as described in Example 2.

### Example 5

The wavelengths and circular dichroism intensities of a circular dichroism spectrometer were calibrated in the same manner as in Example 1. Using the circular dichroism spectrometer, a circular dichroism spectrum of a 1:1 sandwich-bonded complex between a dizinc porphyrin dimer and (R,R)-1,2-diaminocyclohexane as represented by General Formula (VI) shown below was then measured to determine the circular dichroism (Δε). A peak showing a first Cotton effect appeared at 436 nm, and Δε at that peak was −352 cm⁻¹ M⁻¹. A peak showing a second Cotton effect also appeared at 409 nm, and Δε at that peak was +240 cm⁻¹ M⁻¹.

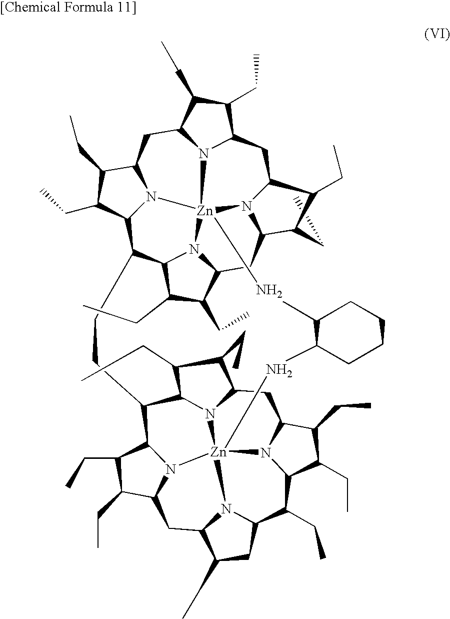

The wavelengths and absorbances of a UV-visible spectrophotometer were then successively calibrated in the same manner as described in Example 2. Using the UV-visible spectrophotometer, a UV-visible absorption spectrum of the complex according to General Formula (VI) was then measured, and the molar extinction coefficients (E) at the wavelengths at which the peaks appeared in the circular dichroism spectrum were determined. At 436 nm, ε was 71,000 cm⁻¹ M⁻¹; and at 409 cm⁻¹, ε was 238,000 cm⁻¹ M⁻¹.

As a result, the anisotropy factor g at the peak wavelength of 436 nm in the circular dichroism spectrum was calculated to be 0.00496, and the anisotropy factor g at the peak wavelength of 409 nm in the circular dichroism spectrum was calculated to be 0.00101.

1. A standard sample for use in testing and/or calibrating a circular dichroism spectrometer and a UV-visible spectrophotometer; the standard sample comprising:
a chlorin dimer represented by Chemical Formula (I): 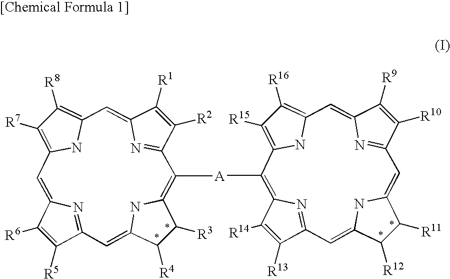
wherein A is CₙH₂ₙ (n is an integer of 0 or more); and R¹, R², R³, R⁴, R⁵, R⁶, R⁷. R⁸, R⁹, R¹⁰, R¹¹, R¹², R¹³, R¹⁴, R¹⁵ and R¹⁶ are the same or different, and are each a hydrogen atom, a saturated hydrocarbon group, an aryl group that may be substituted, a heteroaryl group that may be substituted, or an aralkyl group that may be substituted; or
a metal chlorin dimer represented by Chemical Formula (II): 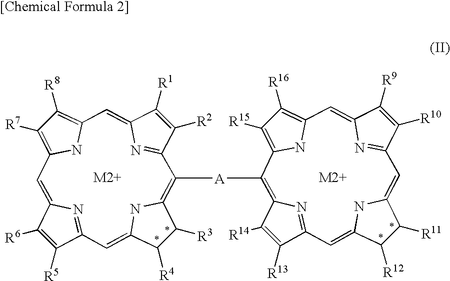
wherein M²⁺ is a divalent metal ion; and A, R¹, R², R³, R⁴, R⁵, R⁶, R⁷, R⁸, R⁹, R¹⁰, R¹¹, R¹², R¹³, R¹⁴, R¹⁵, and R¹⁶ are the same as defined above;
each of the chlorin dimer and the metal chlorin dimer exhibiting at least two peaks in an ultraviolet to visible region of a circular dichroism spectrum and a UV-visible absorption spectrum thereof.

2. The standard sample according to claim 1, wherein R¹, R², R³, R⁴, R⁵, R⁶, R⁷, R⁸, R⁹, R¹⁰, R¹¹, R¹², R¹³, R¹⁴, R¹⁵ and R¹⁶ are the same or different, and are each a C₁-C₂ linear alkyl group or a C₃-C₁₀ linear or branched alkyl group.

3. The standard sample according to claim 1, wherein the chlorin dimer is an (S,S;S,S) chlorin dimer or an (R,R;R,R) chlorin dimer, and the metal chlorin dimer is an (S,S;S,S) metal chlorin dimer or an (R,R;R,R) metal chlorin dimer.

4. The standard sample according to claim 1, wherein the chlorin dimer or the metal chlorin dimer is dissolved in a solvent.

5. The standard sample according to claim 1, wherein M²⁺ is Zn²⁺.

6. A method for producing the standard sample of claim 3, comprising:
(1) a first step of reducing a porphyrin dimer represented by Chemical Formula (III): 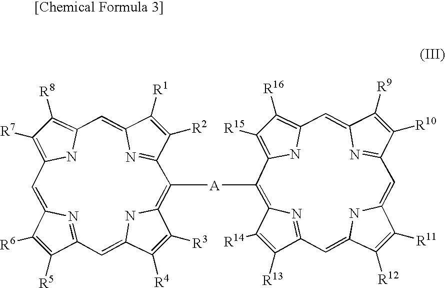
wherein A, R¹, R², R³, R⁴, R⁵, R⁶, R⁷, R⁸, R⁹, R¹⁰, R¹¹, R¹², R¹³, R¹⁴, R¹⁵, and R¹⁶ are the same as defined above; to produce a racemic mixture of an (S, S/S, S) chlorin dimer and an (R, R/R, R) chlorin dimer, represented by Chemical Formula (I): 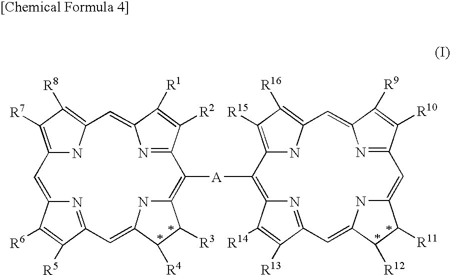
wherein A, R¹, R², R³, R⁴, R⁵, R⁶, R⁷, R⁸, R⁹, R¹⁰, R¹¹, R¹², R¹³R¹⁴, R¹⁵, and R¹⁶ are the same as defined above; and
(2) a second step of optically resolving the resulting racemic mixture to obtain an (S,S;S,S) chlorin dimer or an (R,R;R,R) chlorin dimer.

7. A method for calibrating wavelengths and circular dichroism intensities of a circular dichroism spectrometer using the standard sample of claim 1, the method comprising:
(1) a first step of preparing a chlorin dimer or a metal chlorin dimer whose circular dichroism spectrum has, in order from longer wavelengths:
(a) a wavelength X1 and an intensity Y1 of a peak showing a first Cotton effect;
(b) a wavelength X2 and an intensity Y2 of a peak showing a second Cotton effect;
(c) a wavelength X3 and an intensity Y3 of a peak showing a third Cotton effect; and
(d) a wavelength X4 and an intensity Y4 of a peak showing a fourth Cotton effect;
(2) a second step of measuring a circular dichroism spectrum of the standard sample, using a circular dichroism spectrometer to be calibrated, to determine, in order from longer wavelengths:
(a) a wavelength X′1 and an intensity Y′1 of a peak showing a first Cotton effect;
(b) a wavelength X′2 and an intensity Y′2 of a peak showing a second Cotton effect;
(c) a wavelength X′3 and an intensity Y′3 of a peak showing a third Cotton effect; and
(d) a wavelength X′4 and an intensity Y′4 of a peak showing a fourth Cotton effect; and
(3) a third step of performing at least one process selected from the group consisting of: (a) calibrating the wavelength X′1 and the intensity Y′1 determined at the second step to the wavelength X1 and the intensity Y1, respectively; and (b) calibrating the wavelength X′2 and the intensity Y′2 determined at the second step to the wavelength X2 and the intensity Y2, respectively; and performing at least one process selected from the group consisting of: (c) calibrating the wavelength X′3 and the intensity Y′3 determined at the second step to the wavelength X3 and the intensity Y3, respectively; and (d) calibrating the wavelength X′4 and the intensity Y′4 determined at the second step to the wavelength X4 and the intensity Y4, respectively.

8. A method for calibrating wavelengths and absorbances of a UV-visible spectrophotometer using the standard sample of claim 1, the method comprising:
(1) a first step of preparing a chlorin dimer or a metal chlorin dimer whose UV-visible absorption spectrum has, in order from longer wavelengths:
(a) a wavelength X1 and an absorbance Y1 of a first peak, and
(b) a wavelength X2 and an absorbance Y2 of a second peak;
(2) a second step of measuring a UV-visible absorption spectrum of the standard sample, using a UV-visible spectrophotometer to be calibrated, to determine, in order from longer wavelengths:
(a) a wavelength X′1 and an absorbance Y′1 of a first peak, and
(b) a wavelength X′2 and an absorbance Y′2 of a second peak; and
(3) a third step of calibrating the UV-visible spectrophotometer so that (a) the wavelength X′1 and the absorbance Y′1 and (b) the wavelength X′2 and the absorbance Y′2 determined at the second step become (a) the wavelength X1 and the absorbance Y1 and (b) the wavelength X2 and the absorbance Y2, respectively.

9. A method for calculating an anisotropy factor (g) of a target sample, comprising:
(1) a first step of calibrating wavelengths and circular dichroism intensities of a circular dichroism spectrometer, using the standard sample of claim 1;
(2) a second step of measuring a circular dichroism spectrum of a target sample, using the circular dichroism spectrometer calibrated in the first step, to determine circular dichroism (Δε) at the specific wavelength(s);
(3) a third step of calibrating wavelengths and absorbances of a UV-visible spectrophotometer using the standard sample of claim 1;
(4) a fourth step of measuring a UV-visible absorption spectrum of the target sample, using the UV-visible spectrophotometer calibrated in the third step, to determine a molar extinction coefficient (c) at the same wavelength as in the case of Δε; and
(5) a fifth step of dividing the circular dichroism (Δε) determined in the second step by the molar extinction coefficient (ε) determined in the fourth step to determine an anisotropy factor (g).

10. The method according to claim 9, wherein
(1) the first step of calibrating wavelengths and circular dichroism intensities of a circular dichroism spectrometer; and (2) the second step of measuring a circular dichroism spectrum of the target sample are performed; and then successively, (3) the third step of calibrating wavelengths and absorbances of a UV-visible spectrophotometer; and (4) the fourth step of measuring a UV-visible absorption spectrum of the target sample are performed.

11. The method according to claim 9, wherein
(1) the third step of calibrating wavelengths and absorbances of a UV-visible spectrophotometer; and (2) the fourth step of measuring a UV-visible absorption spectrum of the target sample are performed; and then successively, (3) the first step of calibrating wavelengths and circular dichroism intensities of a circular dichroism spectrometer; and (4) the second step of measuring a circular dichroism spectrum of the target sample are performed.
